(19) United States
(12) Patent Application Publication
(10) Pub. No.: US 2015/0011993 A1
(43) Pub. Date: Jan. 8, 2015

(54) ELECTROSURGICAL PROBE AND ELECTROSURGERY DEVICE

(71) Applicant: OLYMPUS WINTER & IBE GMBH, Hamburg (DE)

(72) Inventor: Andreas Hörlle, Berlin (DE)

(21) Appl. No.: 14/352,206

(22) PCT Filed: Nov. 2, 2012
(86) PCT No.: PCT/EP2012/071737

§ 371 Date: Apr. 16, 2014

(30) Foreign Application Priority Data
Nov. 2, 2011 (DE) 10 2011 085 616.1

(51) Int. Cl. A61B 18/14

(52) U.S. Cl. CPC A61B 18/1492; A61B 2018/00083
USPC 606/41

(57) ABSTRACT

The invention relates to an electrosurgical probe, including a rod-shaped probe body, at least two electrodes, and a fluid outlet, wherein a separating element is provided between the electrodes.

The invention relates to an electrosurgical probe comprising a rod-shaped probe body. The rod-shaped probe body has a surface facing toward its environment. Furthermore, it has a proximal electrode in the form of an electrically conductive portion of the outer surface and, electrically insulated and spaced apart from the proximal electrode in the longitudinal direction of the main body, a distal electrode in the form of a further electrically conductive portion of the outer surface. Moreover, the probe body has a fluid outlet open toward the environment.

The invention further relates to an electrosurgery device having an electrosurgical probe according to the invention, and having a high-frequency (HF) generator, which is connected to the distal electrode and to the proximal electrode and, in operation, supplies a high-frequency voltage.

Electrosurgical probes are used to treat tissue in the body. Typically, they are either bipolar, i.e. they have different electrodes that can be connected to the two poles of an HF generator, or they are alternatively monopolar, i.e. they have just one electrode that can be connected to one pole, which necessitates the additional use of a neutral electrode. For use in some tissue areas, for example of hollow organs, electro-surgical probes can be designed to conform to the tissue.

The document U.S. Pat. No. 7,150,745 B2 discloses an electrode arrangement which is mounted on an expandable member. The document U.S. Pat. No. 7,261,711 B2 discloses a bipolar electrosurgical probe, wherein two electrodes spaced apart from each other are provided, which can be increased in size with the aid of a conductive fluid. The document U.S. Pat. No. 7,959,631 B2 discloses an electro-surgical probe in which tissue can be pressed against electrodes with the aid of an inflatable balloon. The document U.S. Pat. No. 8,012,152 B2 discloses a catheter with a virtual electrode in which conductive hydrogel is used.

It would be desirable for an electrosurgical probe with at least 2 electrodes to be designed in an alternative way, for example more simply and more compact.

According to the invention, this is achieved by an electrosurgical probe according to claim 1 and by an electrosurgery device according to claim 15. Advantageous embodiments can be gleaned, for example, from the dependent claims.

According to a first aspect, the invention relates to an electrosurgical probe comprising a rod-shaped probe body. The probe body can be flexible or stiff. The probe body has an outer surface facing toward its environment. In addition, the probe body has a proximal electrode in the form of an electrically conductive portion of the outer surface and, electrically insulated and spaced apart from the proximal electrode in the longitudinal direction of the main body, a distal electrode in the form of a further electrically conductive portion of the outer surface. In addition, the probe body has an electrically insulating separating element which is arranged between the proximal electrode and the distal electrode and which is expandable transversely with respect to the longitudinal direction, which separating element can preferably be actuated via an actuation mechanism formed in the probe body. With the actuation mechanism, the separating element can be converted from a state in which it is contracted with respect to the longitudinal direction to a state in which it is expanded with respect to the longitudinal direction.

The probe body can have a fluid outlet open toward the environment. Alternatively, however, a fluid outlet can also be provided in another way, for example via an external line which is routed along the probe body. A line to the fluid outlet is preferably flexible.

The electrosurgical probe according to the first aspect of the invention is a bipolar probe suitable for large hollow organs. By means of a conductive fluid, different diameters of electrode and vessel, in which an electrosurgical treatment takes place, can be electrically bridged. Such an electrosurgical probe can be used particularly simply. For this purpose, the probe is, for example, positioned in a hollow organ in such a way that the fluid outlet is located approximately at a location where the treatment is intended to take place. A conductive fluid, for example a polymer-based NaCl gel, is then discharged from a fluid outlet such that it spreads around the probe. The probe and electrodes can then be pulled back a distance, for example by half the longitudinal distance of the electrodes. This has the effect that the tissue to be treated is now located between the two electrodes of the probe and is electrically conductively connected to the electrodes by means of the fluid. Alternatively, however, it can also be pushed forward, or a movement can be dispensed with. Thereafter, the separating element is converted to its expanded state, as a result of which the conductive fluid is divided into two portions electrically insulated from each other, of which each is in electrically conductive contact with one of the electrodes respectively. It is advantageous if the separating element is expanded so far that it adjoins the surrounding tissue. By virtue of this design, it is readily possible to provide two separate areas of conductive fluid which are spaced so far apart from each other that an electrical flux between the electrodes is substantially prevented on account of the fluid. This permits a compact structure of the electrosurgical probe according to the first aspect of the invention.

In terms of its cross section and its dimensions, the rod-shaped probe body is preferably adapted to the tissue that is to be treated. Particularly preferably, the rod-shaped probe body has a round cross section. It is thus advantageously adapted to round hollow organs. The diameter is preferably between 1.6 and 2.0 mm, e.g. 1.8 mm, although thicker or thinner designs are also possible. Alternatively, however, the probe body can also have a different cross section, for example an oval or rectangular cross section.

The proximal electrode and the distal electrode are each designed as electrically conductive portions of the outer surface. For example, they are made from a metal, e.g. aluminum or stainless steel. The electrodes preferably each have a conductive connection, which is routed to an attachment on the electrosurgical probe. In this way, such an electrode can be connected, for example, to a bipolar attachment of a high-frequency (HF) generator.

The probe according to the invention, together with the electrically conductive fluid, permits the treatment of a hollow organ whose internal dimension is greater than the dimension of the probe with the separating element contracted, using a bipolar radiofrequency technique. With the separating element contracted, the probe preferably has a more or less uniform external diameter along its length, at least in the distal portion of the probe, such that in particular the electrodes can have a smaller external diameter than a respective hollow organ to be treated.

The distal electrode and/or the proximal electrode are each preferably designed extending around the rod-shaped probe body. In the case of a rod-shaped probe body with a round cross section, this means that the respective electrode is designed as a ring. In this way, contact can be achieved all the way round with the surrounding electrically conductive fluid, which leads to a particularly good electrical contact. Even if the fluid were to lose the electrical contact to the electrode at one location, an electrical contact at another location suffices to continue to place the electrically conductive fluid under the applied voltage.

The fluid outlet can be formed, for example, by a simple opening in the outer surface of the rod-shaped probe body. Alternatively, it is also possible to provide several openings, or one opening extending around the probe body. For supply with a fluid, the fluid outlet can be connected to a fluid channel which leads to a fluid attachment of the electrosurgical probe. The fluid attachment, e.g. at the proximal end of the probe, can in this case be connected in turn to an external fluid supply device, e.g. a syringe, which delivers a suitable conductive fluid to the fluid attachment and, therefore, also to the fluid outlet.

Alternatively, however, the rod-shaped probe body can also contain a fluid reservoir, which can be arranged, for example, adjacent to the distal end thereof. In this case, the fluid reservoir is preferably designed in such a way that, upon actuation, a fluid contained in it is released through the fluid outlet. The actuation can take place, for example, by the fluid reservoir being mechanically compressed, or, by means of an attachment, being pressurized by delivery of a further fluid or a gas. The design with a fluid reservoir in the probe body affords the advantage that the electrosurgical probe can be supplied and stored complete with the conductive fluid necessary for its use. An external fluid supply device or other measures for delivering a fluid are no longer necessary in this case.

Alternatively, the gel can also be introduced separately, e.g. by means of a hose or a guide sheath, by means of which the probe body is inserted and which at the same time can be used for delivering a fluid.

The insulating separating element, expandable transversely with respect to the longitudinal direction, is preferably designed in such a way that, in the state in which it is contracted with respect to the longitudinal direction, it does not extend above the rest of the probe body in the cross-sectional direction or does so only very slightly. This has the effect that the electrosurgical probe can be inserted without difficulty into the tissue to be treated, without any risk of a protruding separating element damaging or catching on the tissue. Thus, for example, the probe can be brought into position via an endoscope or bronchoscope. The contracted state is therefore understood here as a state with a reduced external diameter. When the separating element is converted to the state in which it is expanded with respect to the longitudinal direction, its cross-sectional area preferably enlarges in an imaginary plane transverse to the longitudinal direction. The above-described and desired separating effect between two areas of the conductive fluid is achieved in this way.

The separating element is preferably designed extending around the rod-shaped probe body. This permits an action of the separating element on all sides. If the probe body is a round probe body, the separating element is also preferably round, and it also preferably has a round cross section both in its contracted state and also in its expanded state, seen in a plane transverse to the longitudinal axis. This permits particularly good adaptation to round hollow organs.

The fluid outlet is preferably arranged adjacent to the distal electrode, to the proximal electrode or to both. This means that the fluid can emerge precisely at the location where it is needed. Depending on the possible particular types of use, for example the treatment of particular organs, the fluid outlet can however be arranged anywhere on the probe body. A preferred position is, for example, also a tip at the distal end of the probe body, in which case the fluid outlet can be located on an axis of a hollow organ to be treated.

According to one embodiment, the separating element is designed as an expandable envelope of the probe body, which envelope is formed from a portion of the outer surface of the probe body. In this case, the separating element can be converted from its contracted state to its expanded state by expansion of the envelope. The ways in which this can be done are described below.

In this case, a chamber closed off with respect to the environment, and delimited at least partially by the expandable envelope, is formed in the separating element and permits a controlled expansion. For example, the closed-off chamber can be toroidal, and the expandable envelope is located on that side of the torus directed toward the environment of the electrosurgical probe. In this case, the expandable envelope must not be able to be delimited in terms of material. Instead, the closed-off chamber can be formed by a continuous, for example toroidal material.

In one embodiment with a chamber closed off with respect to the environment, the actuation mechanism preferably has an actuation fluid channel which is connected hydraulically or pneumatically to the chamber and leads to an actuation fluid attachment open toward the environment, such that the expandable envelope expands transversely with respect to the longitudinal direction when an actuation fluid is delivered to the actuation fluid attachment by means of an external actuation fluid supply device. The actuation fluid channel can be formed in the probe body. In this case, both the actuation fluid channel and also the closed-off chamber are preferably designed such that they are leaktight in respect of the actuation fluid used. When air is used as actuation fluid, this means that they have to be airtight. When a liquid is used as actuation fluid, there should preferably be leaktightness to this liquid.

By means of the actuation fluid attachment, the pressure in the closed-off chamber can be increased by delivery of an actuation fluid, such that the expandable envelope expands. For this purpose, for example, an actuation fluid supply device in the form of an external compressed air source or a fluid pump can be attached to the actuation fluid attachment.

In an alternative embodiment to the design with an actuation fluid channel, the actuation mechanism has a compressing mechanism which, when actuated, causes a compression of the envelope in the longitudinal direction and thus brings about an expansion of the envelope transversely with respect to the longitudinal direction. Here, compression means the reduction of the space available to the expandable envelope in the longitudinal direction. Since the material is compressed in the longitudinal direction but can deflect out toward the environment, the expandable envelope will typically bulge outward when compressed.

Preferably, the expandable envelope, seen in the longitudinal direction, is secured at its distal or proximal end to the compressing mechanism and is secured at the opposite end to a further component of the probe body, such that the expandable envelope expands transversely with respect to the longitudinal direction on account of compression by the compressing mechanism. In this case, the envelope can consist merely of an annular flexible material which, as a component part of the probe body, extends around same. This permits a particularly simple design. Alternatively, however, it is also possible, when using a compressing mechanism, to provide a closed-off chamber in the probe body, which chamber is delimited from the environment by the expandable envelope.

According to an alternative embodiment to the design with an expandable envelope, the separating element is designed as a deployable member which, seen in the longitudinal direction in the non-deployed state, has a distal end and a proximal end and is secured only with either the distal end or the proximal end, whereas the opposite end is a free end. The deployable member in the contracted state forms a portion of the outer surface of the probe body. The actuation mechanism in this case has a compressing mechanism and at least one elongate bending spring which, seen in the longitudinal direction, is secured at the distal or proximal end to the compressing mechanism and, at the opposite end, is secured to a further component of the probe body, such that the bending spring expands transversely with respect to the longitudinal direction on account of compression by the compressing mechanism. The at least one spring, seen from the outside, is arranged under the deployable member, such that the deployable member likewise expands transversely with respect to the longitudinal direction upon expansion of the at least one spring. The spring can also be cast into a material, for example rubber, of the deployable member.

In the design with a deployable member, a similar principle is used as in the design with an expandable envelope and a compressing mechanism. By contrast, however, in the design with a deployable member, it is not the deployable member itself but an elongate bending spring lying underneath it that is caused to expand transversely with respect to the longitudinal direction as a result of compression, whereupon the deployable member lying on top expands together with the bending spring.

The deployable member is preferably designed extending around the probe body. For example, the deployable member has an umbrella-like structure, such that it deploys in a manner similar to an umbrella when the separating element converts from the contracted state to the expanded state.

Several elongate bending springs are preferably applied, thereby permitting a more uniform deployment of the deployable member. For example, several such elongate bending springs are arranged at equal intervals about a circumference of the probe body.

In a further alternative embodiment to the design with an expandable envelope and to the design with a deployable member, the separating element is designed as a twistable member, which forms a portion of the outer surface of the probe body. In this embodiment, the actuation mechanism has a rotary mechanism, wherein the twistable member, seen in the longitudinal direction, is secured at its distal or proximal end to the rotary mechanism and, at its opposite end, is secured to a further component of the probe body, such that the twistable member is twisted upon rotation of the rotary mechanism.

The twistable member is preferably designed such that it bulges out in a rest state. It is also twistable, i.e. it can be turned only at one end, seen in the longitudinal direction, and in so doing experiences torsion. The other end is held secure, which in the present case is achieved by the fact that the twistable member, at its end opposite the rotary mechanism, is secured to a further component of the probe body.

The distance between a point at one end of the twistable member and an opposite further point at the opposite end of the twistable member is increased during torsion of the member. The material lying between these, points which typically bulges out in the rest state, as has been mentioned, must therefore extend a longer distance upon torsion. In this way, the bulging out of the twistable member is reduced. By reducing the torsion, this is accordingly reversed again, such that the bulging out of the twistable member increases again. It is thus possible, by torsion of the twistable member, to control the bulging of the twistable member, as a result of which the separating element can in turn be converted between its contracted state and its expanded state.

The increase or reduction of torsion can be effected with the aid of the rotary mechanism, which can be actuated by a user of the electrosurgical probe. For this purpose, for example, a wheel can be mounted at one end of the electrosurgical probe, with which wheel the rotary mechanism can be turned. However, other designs are also conceivable for this purpose.

According to yet another alternative embodiment, the separating element is designed as a spring basket, which has a number of leaf springs secured at both ends as seen in the longitudinal direction. In this case, the actuation mechanism has a compressing mechanism, wherein the leaf springs, seen in the longitudinal direction, are secured at their distal or proximal end to the compressing mechanism and, at the respective opposite end, are secured to a further component of the probe body, such that the leaf springs expand transversely with respect to the longitudinal direction on account of compression by the compressing mechanism. Otherwise, such an embodiment can be used very similarly to one with an expandable member.

According to a preferred embodiment, the electro-surgical probe has a multiplicity of markings on a proximal end of the rod-shaped probe body, which markings are at a distance from each other, in the longitudinal direction, that corresponds to the longitudinal distance of the electrodes from each other or to half of this distance. The marking can be configured, for example, in the form of a scale. The marking indicates to the user how far the electrosurgical probe has been inserted into the body of the patient or into an organ. The marking is particularly advantageous if, as is intended here, an electrically conductive fluid is used. It is particularly advantageous if the longitudinal distance between the markings corresponds to half the longitudinal distance between the electrodes and, moreover, if the fluid outlet is arranged directly adjacent to one of the electrodes, in particular the proximal electrode. In this case, the electrically conductive fluid can be discharged at a location to be treated in the hollow organ, whereupon it spreads about the environment. The probe is then pulled back until a midpoint between the two electrodes is situated at the location where the fluid was previously discharged and which is intended to be treated.

According to a second aspect, the invention relates to an electrosurgery device having an electrosurgical probe according to the first aspect of the invention, and having an HF generator, which is connected to the distal electrode and to the proximal electrode and, in operation, delivers a high-frequency voltage. The electrosurgery device makes use of the above-described advantages of the electrosurgical probe according to the first aspect of the invention. The embodiment variants and advantages described there also apply to the electrosurgery device according to the second aspect of the invention. According to the second aspect of the invention, the electrosurgery device is to be understood as a unit that can be used directly.

According to a preferred embodiment, the electrosurgery device furthermore has a fluid supply device which is fluidically connected to a fluid outlet, either formed in the probe body or separate therefrom, and in operation supplies the fluid outlet with a conductive fluid. The conductive fluid is, for example, a polymer-containing NaCl gel, which is electrically conductive on account of the ions it contains. The flowability of the gel is determined by the polymer fraction. Compared to a more free-flowing liquid, a viscous gel has the advantage that it does not so easily flow off into hollow organs, e.g. bronchi.

Further advantages and embodiments of the invention will become clear from the illustrative embodiments described below with reference to the figures.

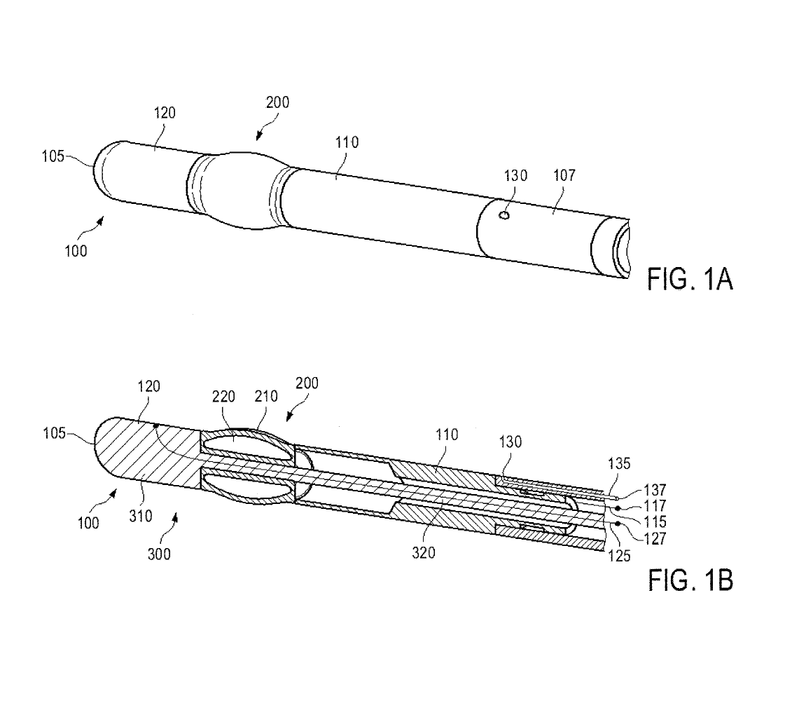

FIG. 1 shows a first illustrative embodiment of an electrosurgical probe according to the first aspect of the invention.

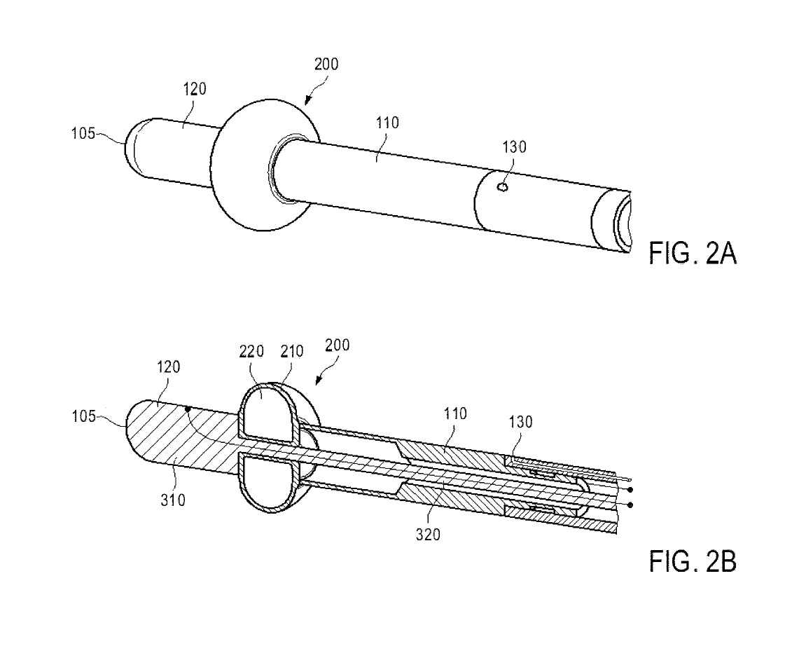

FIG. 2 shows the illustrative embodiment from FIG. 1, with the separating element located in its expanded state.

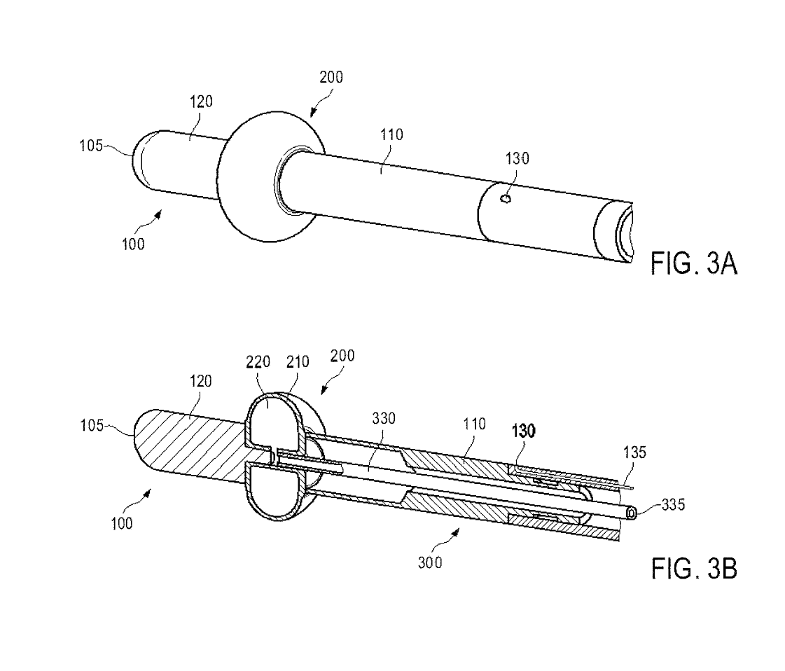

FIG. 3 shows a second illustrative embodiment of the electrosurgical probe according to the first aspect of the invention.

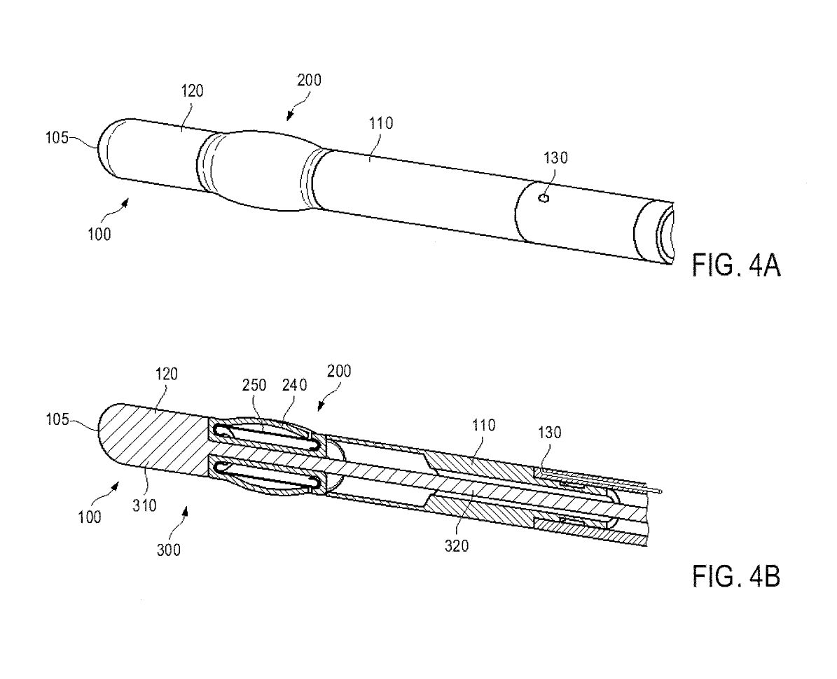

FIG. 4 shows a third illustrative embodiment of an electrosurgical probe according to the first aspect of the invention.

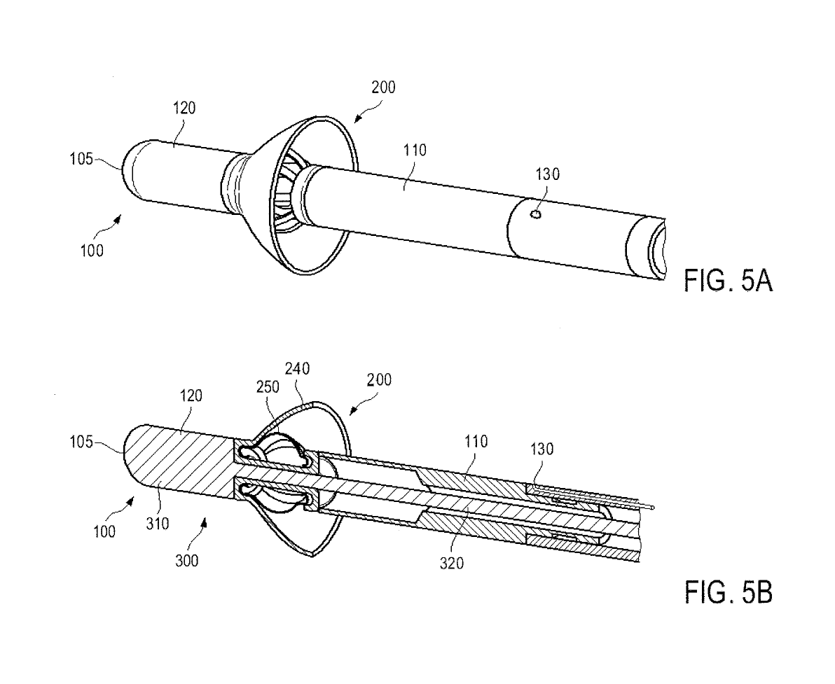

FIG. 5 shows the illustrative embodiment from FIG. 4, with the separating element located in its expanded state.

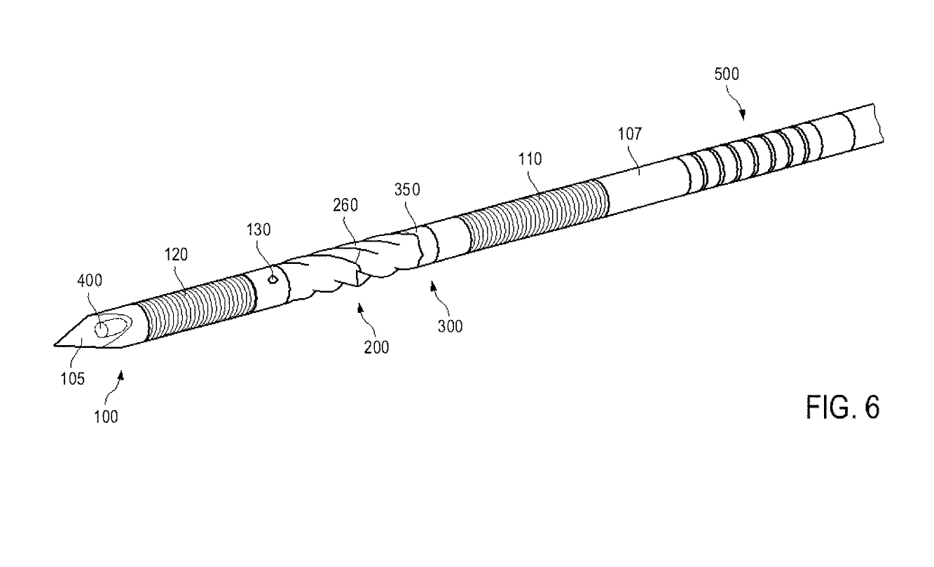

FIG. 6 shows a fourth illustrative embodiment of an electrosurgical probe according to the first aspect of the invention.

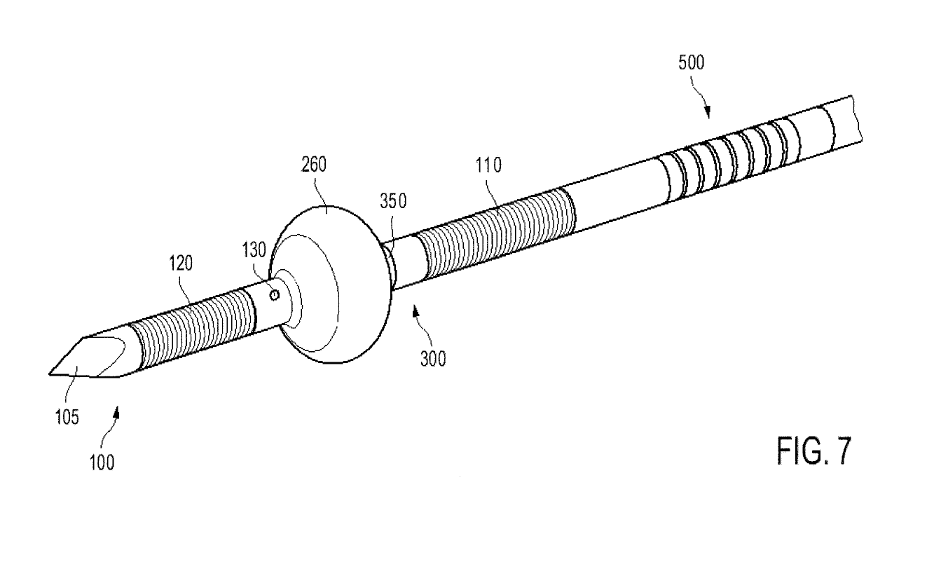

FIG. 7 shows the illustrative embodiment from FIG. 6, with the separating element located in its expanded state.

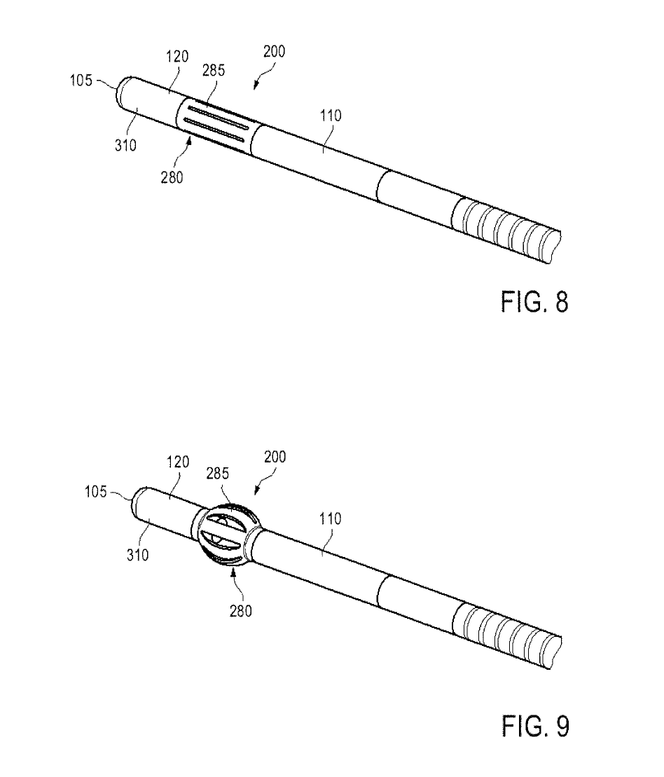

FIG. 8 shows a fifth illustrative embodiment of an electrosurgical probe according to the first aspect of the invention.

FIG. 9 shows the illustrative embodiment from FIG. 8, with the separating element located in its expanded state.

FIG. 10 shows a modification of the illustrative embodiment from FIGS. 1 and 2.

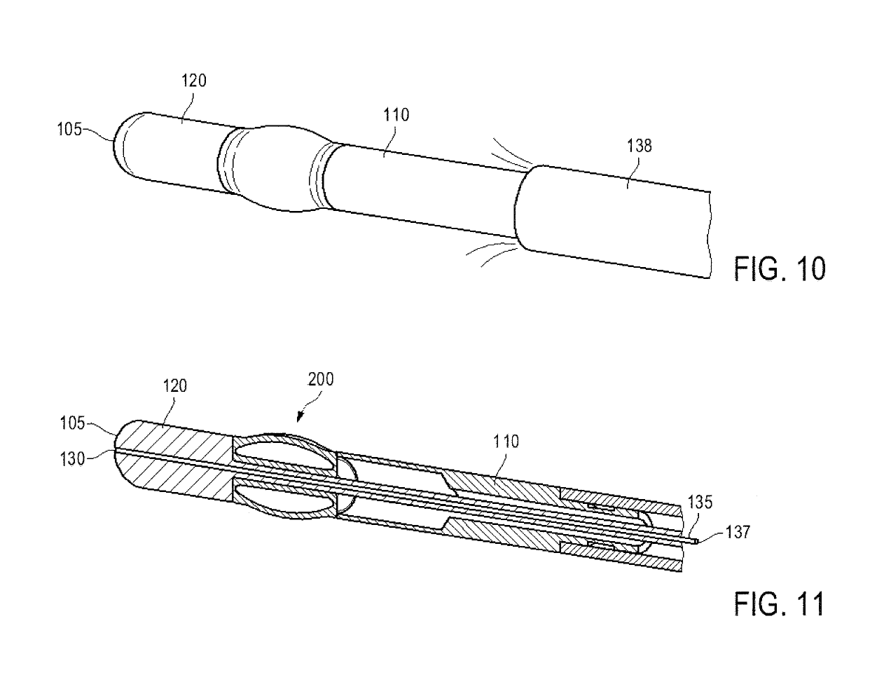

FIG. 11 shows a further modification of the illustrative embodiment of FIGS. 1 and 2.

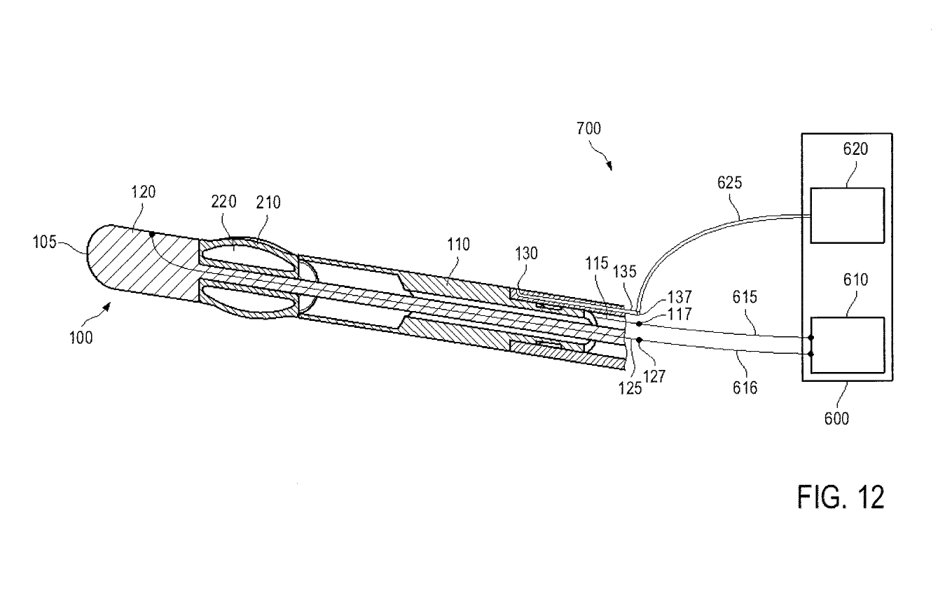

FIG. 12 shows an illustrative embodiment of an electrosurgery device according to the second aspect of the invention.

FIG. 1 shows a first illustrative embodiment of an electrosurgical probe 100 according to the first aspect of the invention. An exterior view of the electrosurgical probe 100 is shown in FIG. 1a, while a cross-sectional view is shown in FIG. 1b. The same applies analogously for FIGS. 2 to 5.

The electrosurgical probe 100 has a probe body 105. The probe body 105 in turn has an outer surface 107 facing toward the environment.

The probe body 105 has a proximal electrode 110 and a distal electrode 120, which are both designed as respective electrically conductive portions of the outer surface 107. The proximal electrode 110 and the distal electrode 120 are designed here extending in a ring shape.

The proximal electrode 110 is connected to a first attachment 117 by means of a first attachment line 115, as a result of which it can be connected to an external voltage source. Likewise, the distal electrode 120 is connected to a second attachment line 125, which is in turn connected to a second attachment 127. In this way, the distal electrode 120 can likewise be connected to an external voltage source. It is preferable if both the proximal electrode 110 and also the distal electrode 120 are connected in operation to a high-frequency (HF) generator, the latter typically having two poles of a bipolar output and thus permitting the attachment of both electrodes.

The probe body 105 furthermore has a fluid outlet 130, which is connected to a fluid attachment 137 by means of a fluid line 135. By way of the fluid attachment 137, it is possible for the fluid line 135, and therefore also the fluid outlet 130, to be connected to an external fluid supply device.

The probe body 105 furthermore has a separating element 200 which, in the present case, is designed in the form of an expandable envelope 210. The expandable envelope 210 encloses a chamber 220 and is made of an electrically non-conductive, electrically insulating material. The chamber can be filled with a gel, a liquid or a gas.

The probe body 105 furthermore has an actuation mechanism 300, which in the present case is formed by a compressing mechanism 310 and a rod 320. The expandable envelope 210 is arranged between the compressing mechanism 310 and the portion of the probe body 105 located proximally in relation to the separating element 200. The compressing mechanism 310 can be moved, by means of the rod 320, in the direction of the proximal portion of the probe body 105. The rod 320 is correspondingly flexible. At the same time, the expandable envelope 210 is pressed together, i.e. compressed. As a result of the elasticity of the expandable envelope 210, the expandable envelope 210 is thus bulged out toward the environment by overpressure.

The rod is preferably a central electrical conductor, and the compressing device is a distal or tip electrode which is electrically connected by the rod to a generator, for example.

A state after the expandable envelope 210 has bulged out is shown in FIGS. 2a and 2b. Otherwise, the embodiments of FIGS. 1 and 2 are identical, and therefore a renewed description of the components is unnecessary.

As can be seen in FIG. 2b, the expandable envelope 210 has bulged outward and is clamped between the compressing mechanism 310 and the portion of the probe body 105 located proximally in relation to the separating element 200.

The expandable envelope 210, bulging out as shown in FIG. 2b, can abut surrounding tissue and thus prevent contact between fluid areas located proximally and distally in relation to the separating element 200. The two fluid areas are thus in contact only with one of proximal electrode 110 and distal electrode 120, respectively, such that a current can flow through tissue located adjacent to the separating element 200, by a voltage difference being applied between the two electrodes 110, 120. The respective electrically conductive fluid between electrode and vessel wall provides a good electrical contact between the respective electrode and the tissue adjacent to the vessel wall. A short circuit between the two electrodes via the electrically conductive fluid is substantially prevented or reduced by the expandable portion of the envelope, which acts an insulator. This permits electrosurgical treatment of this tissue.

FIG. 3 shows a second illustrative embodiment of an electrosurgical probe 100 according to the first aspect of the invention. This is a modification of the illustrative embodiment depicted in FIGS. 1 and 2. Therefore, identical elements having the same function are not dealt with again.

In a modification of the illustrative embodiment of FIGS. 1 and 2, the actuation mechanism 300 in the illustrative embodiment of FIG. 3 has an actuation fluid channel 330 and an actuation fluid attachment 335. The actuation fluid attachment 335 is connected pneumatically or hydraulically to the chamber 220. By introduction of an actuation fluid, for example compressed air or a liquid, the actuation fluid is conveyed through the actuation fluid channel 330 into the chamber 220. The actuation fluid channel 330 is correspondingly flexible. On account of the resulting pressure increase inside the chamber 220 relative to the surrounding pressure, the expandable envelope 210 bulges out, without this requiring compression by a compressing mechanism. For this purpose, the material of the expandable envelope 210 is elastic.

The effect and use of the outwardly bulging envelope 210, as shown in FIG. 3a and FIG. 3b, are identical to those already described with reference to FIG. 2. It is therefore possible here to dispense with further explanation.

FIG. 4 shows a third illustrative embodiment of an electrosurgical probe 100 according to the first aspect of the invention. In a modification of the illustrative embodiment depicted in FIGS. 1 and 2, the probe body 105 of the illustrative embodiment shown in FIG. 4 has no expandable envelope, but instead a deployable member 240 as separating element 200. Other elements are identical and also have an identical function, for which reason they do not have to be described again here.

As in the illustrative embodiment shown in FIG. 1, the electrosurgical probe 100 shown in FIG. 4 has an actuation mechanism 300 with a compressing mechanism 310 and a rod 320. By means of the rod 320, the compressing mechanism 310 can be pulled in the direction of the portion of the probe body 105 located proximally in relation to the separating element 200.

However, unlike the expandable envelope 210 of FIGS. 1 and 2, the deployable member 240 is not secured both at its distal end and also at its proximal end as seen in the longitudinal direction, but only at its distal end. The opposite end, which can also be referred to as the proximal end in the non-deployed state, is by contrast a free end and can move away from the rest of the probe body 105.

Arranged between the compressing mechanism 310 and the portion of the probe body 105 located proximally in relation to the separating element 200, there are several elongate bending springs 250, which are located below the deployable member 240. When the compressing mechanism 310 is pulled in the direction of the proximal end of the probe body 105, the elongate bending springs 250 are pressed together. On account of the compression in the longitudinal direction, they bend outward. Since the deployable member 240 is located directly above the elongate bending springs 250, it too is pressed outward, as a result of which it deploys similarly to an umbrella.

The state thereby reached is shown in FIG. 5. The deployable member 240 is now located in its deployed state, which means that its free end juts out from the rest of the probe body 105. It thus achieves the same effect as the expandable envelope 210 which is shown in the illustrative embodiments of FIGS. 1 to 3. In particular, it can separate two fluid areas from each other.

FIG. 6 shows a fourth illustrative embodiment of an electrosurgical probe 100 according to the first aspect of the invention. In a modification of the illustrative embodiment depicted in FIGS. 1 and 2, the electrosurgical probe 100 of FIG. 6 has no expandable envelope 210, and instead it has a twistable member 260 as separating element 200. Likewise, the actuation mechanism 300 is correspondingly of a different design. Further elements having an identical function are not described again below.

At its distal end as seen in the longitudinal direction, the twistable member 260 is mounted on a component of the probe body 105. At its proximal end, the twistable member 260 is mounted on a rotary device 350 which, in this illustrative embodiment, is a component part of the actuation mechanism 300. The rotary mechanism 350 can be rotated by means of a wheel lying outside the area shown. In this way, the proximal end of the twistable member 260 can also be rotated.

By virtue of the non-rotatable securing of the twistable member 260 at the distal end and the rotatable securing of the twistable member 260 at the proximal end, the twistable member can be twisted with the aid of the rotary mechanism 350. The twistable member 260 is designed such that, in a non-twisted state, i.e. relaxed state, it bulges out. Such a state is shown in FIG. 7. Otherwise, the view in FIG. 7 is identical to that in FIG. 6.

If the twistable member 260 is now twisted by means of the rotary mechanism 350, the bulge of the twistable member 260 disappears, because the distance between opposite points in the longitudinal direction increases during twisting. The twisted state is shown in FIG. 6. Here, the twistable member 260 has drawn inward to such an extent that it no longer protrudes from the other portions of the outer surface 107. In this way, the electrosurgical probe 100 can be inserted into tissue that is to be treated.

In a further modification of the illustrative embodiment of FIGS. 1 and 2, the fluid outlet 130 in the illustrative embodiment of FIG. 6 is not arranged proximally in relation to the separating element 200, but distally in relation thereto. Moreover, the fluid outlet 130 is not connected to a fluid attachment by means of a fluid channel, but is instead connected to a fluid reservoir 400 located in the interior of the probe body 105, specifically at the distal end thereof. In this way, a suitable conductive fluid, for example a polymer-based NaCl gel, can already be kept ready in the probe, which means that it is not necessary to provide an external fluid supply device.

By way of a line not shown here, the fluid reservoir 400 can be pressurized for actuation. In this way, the fluid contained in it is released through the fluid outlet 130.

Furthermore, the illustrative embodiment of FIG. 6 has a scale 500 with a multiplicity of markings. The markings serve to indicate to the user how far the electrosurgical probe 100 has been inserted into the tissue to be treated. In addition, when moving the electrosurgical probe 100 after the discharge of the fluid, the markings help to position the separating element adjacent to the tissue to be treated.

FIG. 8 shows a fifth illustrative embodiment of an electrosurgical probe 100 according to the first aspect of the invention. In a modification of the illustrative embodiment depicted in FIGS. 4 and 5, the electrosurgical probe 100 of FIG. 8 has no deployable member 240, but it instead has a spring basket 280 as separating element 200. The actuation mechanism 300 is of identical design to the one shown in FIGS. 4 and 5. Other elements having an identical function are also not described again below.

The spring basket 280 has a multiplicity of leaf springs 285 which, seen in the longitudinal direction, are secured at both ends, specifically at one end on the compressing mechanism 310 and at the other end on a further part of the probe body 105. In the same way as the bending springs 250 shown in FIGS. 4 and 5, it is thus possible for the leaf springs 285 to be pressed outward by pulling the compressing mechanism 310 in the direction of the proximal end of the probe body 105. In this way, the separating element 300 is converted to its expanded state, which is shown in FIG. 9.

In a further modification of the previous illustrative embodiments, the probe body 105 of the fifth illustrative embodiment has no fluid outlet. It is thus designed to be used together with an external fluid supply, for example a hose.

FIG. 10 shows an illustrative embodiment of a probe body 105 which is designed similarly to that of FIGS. 1 and 2. However, in a modification thereof, it has a guide sheath 138, which annularly encloses the proximal end of the probe body 105. The guide sheath 138 is designed in the form of a hose, of which the diameter is greater than the diameter of the probe body 105. Thus, a space through which a fluid can be delivered remains between the probe body 105 and the guide sheath 138. The guide sheath 138 can also be used for holding the probe body 105 and inserting it into the tissue to be treated.

FIG. 11 shows a cross-sectional view of an illustrative embodiment of a probe body 105 which is likewise of a design similar to that of FIGS. 1 and 2, but which has a different position of the fluid outlet 130. In the illustrative embodiment of FIG. 11, the fluid outlet 130 is located on the tip at the distal end of the probe body 105. Accordingly, the fluid line 135 extends from the fluid inlet 137 to the tip of the probe body 105. With the arrangement of the fluid outlet 130 as shown in FIG. 11, the fluid can emerge at the tip of the probe body 105. If the probe body is located more or less centrally, for example, in a surrounding round hollow organ, the fluid can thus emerge at a location from which it has similar distances to travel to the surrounding tissue of the hollow organ. A more uniform distribution of the fluid can thus be achieved.

FIG. 12 shows an illustrative embodiment of an electrosurgery device 700 according to the second aspect of the invention. The electrosurgery device 700 has an electrosurgical probe 100 which is identical to the one that was described with reference to FIGS. 1 and 2. It is therefore possible to omit repeated mention of similar elements.

The electrosurgery device 700 also has a supply unit 600, which in turn has an HF generator 610 and a fluid supply device 620. The HF generator 610 is connected by means of two lines 615, 616 to the first attachment 117 and to the second attachment 127 of the electrosurgical probe 100. Thus, the HF generator 610 can supply the proximal electrode 110 and the distal electrode 120 with high-frequency voltage, as is necessary for bipolar electrosurgical treatment.

The fluid supply device 620 is connected by means of a fluid line 625 to the fluid attachment 137 of the electrosurgical probe 100. In this way, the fluid supply device 620 can deliver a fluid to the fluid supply attachment 137 and therefore also to the fluid outlet 130. The fluid supply device 620 thus delivers the fluid that is intended to emerge from the fluid outlet 130.

The fluid supply device 620 is in the present case designed such that it delivers a polymer-based NaCl gel as conductive fluid. For example, a syringe can be used for this purpose.

The described illustrative embodiments of an electrosurgical probe 100, particularly when used as a component part of an electrosurgery device 700, can be employed for the electrosurgical treatment of tissue, e.g. in the lungs, in the bronchi or in veins. For this purpose, the electrosurgical probe 100 can, for example, be placed in a hollow organ in such a way that the fluid outlet 130 is located adjacent to a tissue portion to be treated. In this position, a conductive fluid in the form of a polymer-based NaCl gel is discharged from the fluid outlet 130. This fluid spreads around the probe body 105, thereby coming into contact on the one hand with the probe body 105 and on the other hand with the tissue that is to be treated. Alternatively, another conductive gel could also be used. However, it may happen that the probe body is not yet located in the correct position for treating the tissue at the desired location. In this case, the electrode then has to be shifted such that the separating element 200 is located adjacent to the tissue to be treated. Since the tissue is usually rougher than the outer surface 107 of the probe body 105, the electrically conductive fluid will remain substantially in its place, which is an advantage of the gel.

The fluid now comes into contact with the distal electrode 110, with the proximal electrode 120 and with the surrounding tissue. Since the fluid is conductive, an electrical connection is also established in this way between these elements. In this state, however, a direct electrically conductive connection would also be established between the proximal electrode 110 and the distal electrode 120 via the fluid, such that some of the current flows through the fluid directly from one electrode to the other, for which reason too small a fraction of the current would flow through the tissue to be treated.

To prepare the electrosurgical probe 100 for the treatment of the tissue, the separating element is converted to its expanded state. This is done as has been described in the respective illustrative embodiment. The separating element 200 presses against the surrounding tissue of the hollow organ, such that the electrically conductive fluid, which is located as described between the probe body 105 and the surrounding tissue, is divided into two parts, which are no longer, or only slightly, electrically conductively connected to each other. The separating element 200 consists of an insulator, such that no electrically conductive connection arises via the separating element 200 either. Each of the two electrodes 110, 120 is now electrically conductively connected to a part of the tissue situated laterally from the tissue to be treated, such that, when a voltage is applied between the two electrodes 110, 120, a current flows through the tissue to be treated.

1. An electrosurgical probe,
comprising a rod-shaped probe body,
which has an outer surface facing toward its environment, and
which has a proximal electrode in form of an electrically conductive portion of the outer surface and, electrically insulated and spaced apart from the proximal electrode in the longitudinal direction of the main body, a distal electrode in form of a further electrically conductive portion of the outer surface,
wherein
the probe body comprises an electrically insulating separating element which is arranged between the proximal electrode and the distal electrode and which is expandable transversely with respect to the longitudinal direction,
wherein the separating element can be transferred from a state in which said separating element is contracted with respect to the longitudinal direction to a state in which said separating element is expanded with respect to the longitudinal direction.

2. The electrosurgical probe according to claim 1,
which furthermore comprises a fluid outlet open toward the environment.

3. The electrosurgical probe according to claim 2,
in which the fluid outlet is arranged adjacent to said distal electrode, said proximal electrode or both.

4. The electrosurgical probe according to claim 1,
in which the separating element is designed as an expandable envelope of the probe body, which envelope is formed from a portion of the outer surface of the probe body,
and wherein the separating element is converted from said contracted state to said expanded state by expansion of the envelope.

5. The electrosurgical probe according to claim 4,
wherein a chamber closed off with respect to the environment, and delimited at least partially by the expandable envelope, is formed in the separating element.

6. The electrosurgical probe according to claim 5,
which furthermore comprises an actuation mechanism with an actuation fluid channel which is connected hydraulically or pneumatically to the chamber and leads to an actuation fluid attachment open toward the environment, such that the expandable envelope expands transversely with respect to the longitudinal direction when an actuation fluid is delivered to the actuation fluid attachment.

7. The electrosurgical probe according to claim 4,
which furthermore comprises an actuation mechanism with a compressing mechanism which, when actuated, causes a compression of the envelope in the longitudinal direction and thus brings about an expansion of the envelope transversely with respect to the longitudinal direction.

8. The electrosurgical probe according to claim 7,
in which the expandable envelope, seen in the longitudinal direction, is secured at its distal or proximal end to the compressing mechanism and is secured at the opposite end to a further component of the probe body, such that the expandable envelope expands transversely with respect to the longitudinal direction on account of compression by the compressing mechanism.

9. The electrosurgical probe according to claim 1,
in which the separating element is designed as a deployable member which, seen in the longitudinal direction in the non-deployed state, has a distal end and a proximal end and is secured only with either the distal end or the proximal end, whereas the opposite end is a free end,
wherein the deployable member in the contracted state forms a portion of the outer surface of the probe body,
wherein the probe body furthermore has an actuation mechanism with a compressing mechanism and at least one elongate bending spring which, seen in the longitudinal direction, is secured at the distal or proximal end to the compressing mechanism and, at the opposite end, is secured to a further component of the probe body, such that the bending spring expands transversely with respect to the longitudinal direction on account of compression by the compressing mechanism,
and wherein the at least one spring, seen from the outside, is arranged under the deployable member, such that the deployable member likewise expands transversely with respect to the longitudinal direction upon expansion of the at least one spring.

10. The electrosurgical probe according to claim 1,
in which the separating element is designed as a twistable member, which forms a portion of the outer surface of the probe body,
and in which the actuation mechanism has a rotary mechanism, wherein the twistable member, seen in the longitudinal direction, is secured at its distal or proximal end to the rotary mechanism and, at its opposite end, is secured to a further component of the probe body, such that the twistable member is twisted upon rotation of the rotary mechanism.

11. The electrosurgical probe according to claim 1,
in which the separating element is designed as a spring basket with a multiplicity of leaf springs, which spring basket forms a portion of the outer surface of the probe body,
and in which the probe body furthermore has an actuation mechanism with a compressing mechanism, wherein the leaf springs, seen in the longitudinal direction, are secured at the distal or proximal end to the compressing mechanism and, at the respective opposite end, are secured to a further component of the probe body, such that the leaf springs expand transversely with respect to the longitudinal direction on account of compression by the compressing mechanism.

12. The electrosurgical probe according to claim 1.
in which the rod-shaped probe body comprises a round cross section.

13. The electrosurgical probe according to claim 1,
in which the distal electrode and/or the proximal electrode and/or the separating element are formed all round the rod-shaped probe body.

14. The electrosurgical probe according to claim 1,
which furthermore comprises, adjacent to the distal end of the rod-shaped probe body, a fluid reservoir which, upon actuation, releases a fluid contained therein through a fluid outlet.

15. The electrosurgical probe according to claim 1,
which furthermore comprises a multiplicity of markings on a proximal end of the rod-shaped probe body, which markings are at a distance from each other, in the longitudinal direction, that corresponds to the longitudinal distance of the electrodes from each other or to half of this distance.

16. An electrosurgery device comprising
an electrosurgical probe according to claim 1,
and an HF generator, which is connected to the distal electrode and to the proximal electrode and, in operation, delivers a high-frequency voltage.

17. The electrosurgery device according to claim 16,
in which the electrosurgical probe comprises a fluid outlet,
and which furthermore comprises a fluid supply device which is fluidically connected to the fluid outlet and, in operation, supplies the latter with a conductive fluid, for example a polymer-containing NaCl gel.
